(19) United States
(12) Patent Application Publication
(10) Pub. No.: US 2018/0280207 A1
(43) Pub. Date: Oct. 4, 2018

(54) DISPOSABLE ABSORBENT ARTICLE INCLUDING A LEAK BARRIER

(71) Applicant: H.B. Fuller Company, St. Paul, MN (US)

(72) Inventors: Michael W. Harris, Shoreview, MN (US); Kevin P. Davis, Woodbury, MN (US); Ameara S. Mansour, Woodbury, MN (US); Mark S. Kroll, Arden Hills, MN (US)

(21) Appl. No.: 15/945,470

(22) Filed: Apr. 4, 2018

Related U.S. Application Data
(60) Provisional application No. 62/481,259, filed on Apr. 4, 2017.

(51) Int. Cl. A61F 13/475; A61F 13/534; A61F 13/494; A61F 13/539; A61F 13/15; A61F 13/00; A61L 15/58; A61L 15/24; A01K 1/015; B32B 7/12

(52) U.S. Cl. CPC A61F 13/475; B32B 2555/02; A61F 13/494; A61F 13/49466; A61F 13/539; A61F 13/15739; A61F 13/00068; A61L 15/58; A61L 15/24; A01K 1/0157; B32B 7/12; A61F 2013/5395; A61F 2013/5349; B32B 2255/26; B32B 2307/726; B32B 2250/03; A61F 13/534

(57) ABSTRACT

The invention features a disposable absorbent article including a thermoplastic composition applied in a full coat layer (i.e. a layer with 100% coverage) as a discrete shape to either the top sheet or to a component of the absorbent core (i.e. core or core wrap). The discrete shape is strategically located to decrease the likelihood of leaks in the desired location. The thermoplastic composition serves as a leak barrier to prevent bodily fluids and solids (i.e. urine, stool, menstrual flow, wound seepage) from leaking from the absorbent core.

This application claims the benefit of U.S. Provisional Application No. 62/481,259 filed Apr. 4, 2017, which is incorporated herein.

### BACKGROUND

In the area of industrial adhesives, hot melt adhesives are commonly used to bond together a wide variety of articles including disposable absorbent articles comprising non-woven substrates e.g. adult incontinence products (e.g. briefs and pads), disposable diapers, sanitary napkins, bed pads, puppy pads, medical dressings, etc.

There can be multiple hot melt adhesives used in the manufacture of a disposable absorbent article. For example, in the manufacture of a disposable diaper, adhesives are used in construction (e.g. bonding the back sheet to the top sheet and optionally the absorbent core), elastic attachment (e.g. bonding the elastic material to the back sheet in for example the leg or waist area) and for the core stabilization (e.g. applying an adhesive to the absorbent core to increase the strength of the core).

Modern day disposable absorbent articles are fairly effective. However, there can still be leaks from a disposable absorbent article, especially when the user is lying down. Leaks can cause contaminated clothing and bed coverings, which results in discomfort for the end user and increases the work load of caregivers.

Various design elements have been added to disposable absorbent articles to prevent leaks e.g. wings, improved hook and loop/elastic designs, attached sections (i.e. standing leg cuffs) on the outer edge of the core, etc. Each of these design elements increases the complexity of the disposable article.

There is a need in the industry for a simple, effective way of decreasing disposable absorbent article leaks.

### SUMMARY

In one aspect, the invention features a disposable absorbent article, including a top sheet, a back sheet, and an absorbent core disposed between the top sheet and the back sheet, the top sheet, the back sheet, and the absorbent core forming a layered assembly held together with an attachment means, the disposable absorbent article further including a thermoplastic composition applied in a full coat layer as a discrete shape to the top sheet or to a component of the absorbent core and overlapping a portion of the absorbent core.

In one embodiment, the thermoplastic composition overlaps a component of the absorbent core with direct contact. In another embodiment, the thermoplastic composition is applied to the core facing side of the top sheet.

In a different embodiment, the absorbent core includes a core, and a core wrap, and the thermoplastic composition applied to the core wrap prior to wrapping the core.

In one embodiment, the attachment means is a hot melt adhesive composition. In another embodiment, the disposable absorbent article is selected from the group including an adult incontinence product, disposable diaper, sanitary napkin, bed pad, puppy pad and a medical dressing. In another embodiment, the disposable absorbent article further includes a front waist region, a back waist region, and a crotch region disposed between the front waist region and the back waist region. In one embodiment, the disposable absorbent is selected from the group including an adult incontinence brief and a disposable diaper.

In one embodiment, the thermoplastic composition is applied to the absorbent core facing side of the top sheet. In another embodiment, the thermoplastic composition overlaps a portion of one side of the absorbent core. In a different embodiment, the thermoplastic composition is applied at a coat weight of from about 5 to about 40 gsm. In one embodiment, the hot melt adhesive composition and the thermoplastic composition are the same.

In one embodiment, the thermoplastic composition comprises a polymer selected from the group including a styrene block copolymer and an olefin polymer. In a different embodiment, the thermoplastic composition includes at least 90% by weight of an olefin polymer. In another embodiment, the thermoplastic composition has a viscosity of from about from about 500 to about 25,000 cP at 177° C. In one embodiment, the thermoplastic composition comprises a rectangular shape overlapping a portion of the back waist region of the core.

In one aspect, the invention features a method of forming a leak barrier on a disposable absorbent article including the steps of applying a thermoplastic composition in a full coat layer of a discreet shape to the top sheet or to a component of the absorbent core, and joining the top sheet to a back sheet while inserting the absorbent core, such that the thermoplastic composition overlaps a portion of the absorbent core.

In one embodiment, the disposable absorbent article is selected from the group including an adult incontinence brief and a disposable diaper. In another embodiment, the discreet shape of a thermoplastic composition is applied in molten form with a slot coater.

In another aspect, the invention features an absorbent core including a core, a core wrap, and a thermoplastic composition, wherein the thermoplastic composition is applied in a full coat layer of a discreet shape to the core wrap, so as to directly overlap a portion of the core when wrapped around the core.

The invention features a disposable absorbent article including a thermoplastic composition applied in a full coat layer (i.e. a layer with 100% coverage) as a discrete shape to either the top sheet or to a component of the absorbent core (i.e. core or core wrap) and overlapping a portion of the absorbent core. The discrete shape is strategically located to act as a barrier, preventing bodily fluids and solids (i.e. urine, stool, menstrual flow, wound seepage) from leaking out of the absorbent core.

### DETAILED DESCRIPTION

### Brief Description of The Drawings

FIG. 1 is a disposable absorbent article with the thermoplastic composition applied as a leak barrier to the top sheet to overlap all sides of the core in the finished article.

FIG. 2 is an absorbent core including a core and a core wrap where the thermoplastic composition is applied to the core wrap. The core wrap is wrapped around the core in the finished absorbent core.

### DISPOSABLE ABSORBENT ARTICLE

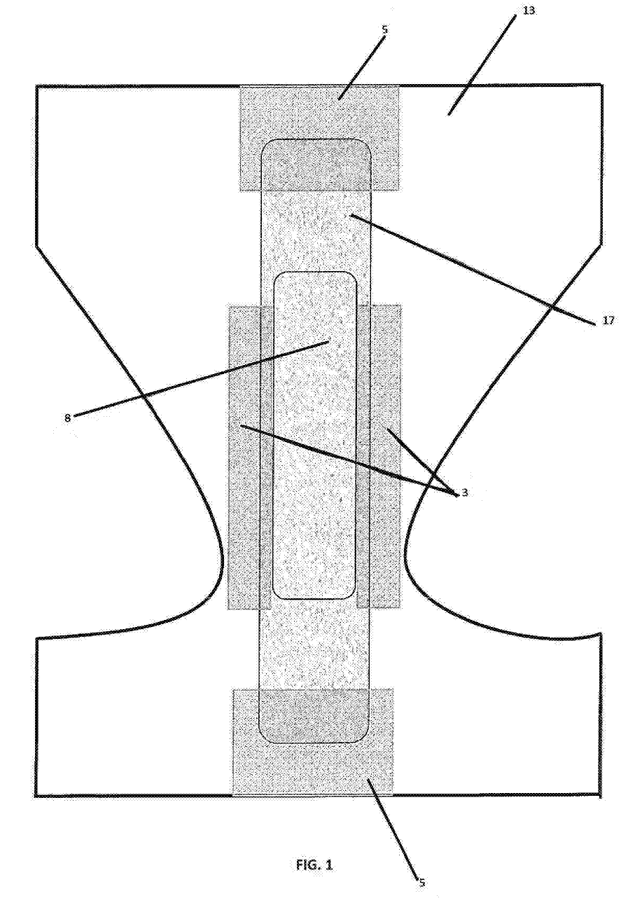

The invention features a disposable absorbent article, e.g. FIG. 1 including a layered assembly 13 that includes a liquid-permeable inner layer or top sheet, a liquid-impermeable outer layer or back sheet, and an absorbent core 17 disposed between. The layered assembly further includes an attachment means to hold the layered assembly together and a thermoplastic composition 5, 3 applied in a full coat layer as a discrete shape. The thermoplastic composition can be applied to the top sheet (FIG. 1) or to a component of the absorbent core. In either case, the thermoplastic composition overlaps a portion of the absorbent core.

The disposable absorbent article can be selected from the group consisting of adult incontinence products (e.g. briefs and pads), disposable diapers, sanitary napkins, bed pads, puppy pads and medical dressings.

### Back Sheet

The back sheet is liquid-impermeable and makes up the outer layer of the disposable article. The back sheet may be constructed from a number of different materials, or may be a composite of several different materials (e.g. a nonwoven could be laminated to the liquid-impermeable layer). In one embodiment, the back sheet is constructed from a polyolefin film. The film may or may not be vapor-permeable. For purposes of description, the term “back sheet” or “outer layer,” as used herein, may refer to any sheet, layer or composite that covers at least the core, but in the case of an adult incontinence brief or diaper can extend beyond the core toward the back waist and fastening (i.e. tab) areas of the article.

### Absorbent Core

The absorbent core includes the core and optionally a core wrap. When the thermoplastic composition is applied to the absorbent core, it can be applied to the core or the core wrap. The core can vary in design, shape, and materials used. It can be shaped like a long rectangle (FIG. 1, 17) or like a dog bone. The core is generally centered within the article, and is firmly secured between the top sheet and back sheet through an attachment means. The core includes absorbent materials able to absorb bodily fluids and solids received through the top sheet. Absorbent materials can include fluff (e.g. cellulose based), super absorbent (e.g. super absorbent particles), nonwoven, tissue, foam, or any other absorbent material.

The core wrap can be any material that wraps around the core to hold the components of the core in place. The core wrap generally wraps around the two longer sides of the core, leaving the ends open. The core wrap can be selected from the group consisting of nonwoven and tissue.

### Acquisition Layer

The disposable article can include an acquisition layer (e.g. FIG. 1, 8). The acquisition layer is a layer of material between the core and the top sheet and assists in directing the bodily fluids and solids into the core. The acquisition layer can be a hydrophilic treated nonwoven, an apertured film, or any other material that can assist in directing bodily fluids and solids into the core.

### Top Sheet

The top sheet is liquid-permeable and makes up the inner layer of the disposable article. For purposes of description, the term “top sheet” or “inner layer,” can refer to any sheet, layer or composite that covers at least the core, but in the case of an adult incontinence brief or diaper can extend beyond the core toward the back waist and the fastening (i.e. tab) areas of the article.

The top sheet may be constructed from a wide range of suitable materials including apertured films, nonwoven webs of natural fibers (e.g., wood or cotton) or synthetic fibers (e.g., polypropylene or polyester) or a combination of such fibers.

The top sheet can be hydrophobic or hydrophilic (e.g. surfactant treated). The top sheet can be hydrophobic, with the exception of the area in the core which can be treated with surfactant to be hydrophilic.

### Attachment Means

The attachment means securely holds together the top sheet and the back sheet, keeping the absorbent core in place between them. The attachment means is not limited and can include any means that will perform the required function. The attachment means can be selected from the group consisting of ultrasonic bonds, thermal bonds, mechanical bonds (e.g. embossing), sewing, and adhesive (e.g. hot melt adhesive). The hot melt adhesive can be a hygiene construction adhesive.

### Thermoplastic Composition

The thermoplastic composition can have a viscosity of less than about 100,000 centipoise (cP) at 177° C., less than about 50,000 cP at 177° C., less than about 25,000 cP at 177° C., from about 500 to about 25,000 cP at 177° C., or even from about 500 cP to about 10,000 cP at 177° C.

The thermoplastic composition can be any typical hygiene hot melt adhesive including those used for construction. For manufacturing ease, the attachment means and the thermoplastic composition can be the same hot melt adhesive.

The thermoplastic composition can comprise a polymer selected from a group consisting of an olefin polymer and a styrene block copolymer.

The olefin polymer can include single-site (e.g. metallocene) catalyzed polyolefins, amorphous poly-alpha olefins (APAO's), polyethylene homopolymers/copolymers and polypropylene homopolymers/copolymers and blends thereof.

Useful propylene homopolymers include L-MODU 400S and L-MODU 600S available from Idemitsu. Kosan Co., Ltd. (Japan). Useful propylene copolymers include VISTAMAXX 6202, VISTAMAXX 8780 and VISTAMAXX 8380 (propylene/ethylene copolymers) available from ExxonMobil Chemical (Houston, Tex.), LICOCENE PP1602 and LICOCENE PP2602 both available from Clariant international Ltd. (Muttenz, Switzerland), REXTAC 2730 (an amorphous poly-alpha olefin) available from Rextac LLC (Odessa, Tex.), and the AERAFIN series of polymers including AERAFIN 180 (an amorphous poly-alpha olefin) available from Eastman Chemical Company (Kingsport, Tenn.). Useful commercially available ethylene interpolymers are available under a variety of trade designations including, e.g. the AFFINITY and ENGAGE series of trade designations from DowDuPont Chemical Company (Midland, Mich.) including, e.g. AFFINITY GA 1900, AFFINITY GA 1950, and ENGAGE 8200.

The styrene block copolymer can include an aromatic vinyl polymer block and a conjugated diene polymer block, a hydrogenated conjugated diene polymer block, or a combination thereof. The blocks can be arranged in a variety of configurations including, e.g., linear, branched, radial, star and combinations thereof. The aromatic vinyl polymer block can be derived from a variety of aromatic vinyl compounds including, e.g., styrene, alpha-methylstyrene, beta-methylstyrene, o-, m-, p-methylstyrene, t-butylstyrene, 2,4,6-trimethyistyrene, monofluorostyrene, difluorostyrene, monochlorostyrene, dichlorostyrene, mcthoxystyrene, 1,3-vinylnaphthalene, vinylanthracene, indene, acenaphthylene, and combinations thereof. The diene polymer block can be derived from a variety of diene-containing compounds including, e.g., isoprene, butadiene, hexadiene, 2,3-dimethyl-1,3-butadiene, 1,3-pentadiene, and hydrogenated versions thereof, and combinations thereof.

Useful styrene block copolymers include, e.g., triblock, multi-arm, and radial copolymers including, e.g., styrene-butadiene-styrene (SBS), styrene-isoprene-styrene (SIS), styrene-butadiene-isobutylene-styrene (SBBS), styrene-isoprene-butadiene-styrene (SIBS), styrene-ethylene/butene-styrene (SEBS), styrene-ethylene/propylene-styrene (SEPS), styrene-ethylene-ethylene/propylene-styrene (SEEPS), and combinations thereof.

Useful styrene block copolymers include VECTOR 4411 and VECTOR 6241 available from TSRC Corporation (Houston, Tex.) and SOLPRENE 411 (a high molecular weight radial SBS block copolymer) available from Dynasol (Houston, Tex.).

In one embodiment, the thermoplastic composition consists of one or more polymers and no greater than 2% additives.

In one embodiment, the thermoplastic composition comprises at least 40% by weight, at least 50% by weight, at least 75% by weight, at least 90% by weight, at least 95% by weight, from about 10% by weight to about 100% by weight, or even from about 20% by weight to about 75% by weight of one or more polymers.

The thermoplastic composition can further comprise additional materials such as e.g. tackifying agents, waxes, plasticizers, fillers and additional additives (e.g. antioxidant, wetness indicator, UV stabilizers, etc.). The thermoplastic composition can comprise less than about 25% by weight of the additional components. The thermoplastic composition can be colored (i.e. can include a pigment or a dye).

### Method of Forming a Leak Barrier

The invention further includes a method of forming a leak barrier on a disposable absorbent article including the steps of applying a thermoplastic composition in a full coat layer of a discreet shape to the top sheet or to a component of the absorbent core (i.e. core or core wrap), and joining the top sheet to the back sheet while inserting the absorbent core; such that the thermoplastic composition overlaps a portion of the absorbent core.

The thermoplastic composition can overlap the absorbent core directly or indirectly.

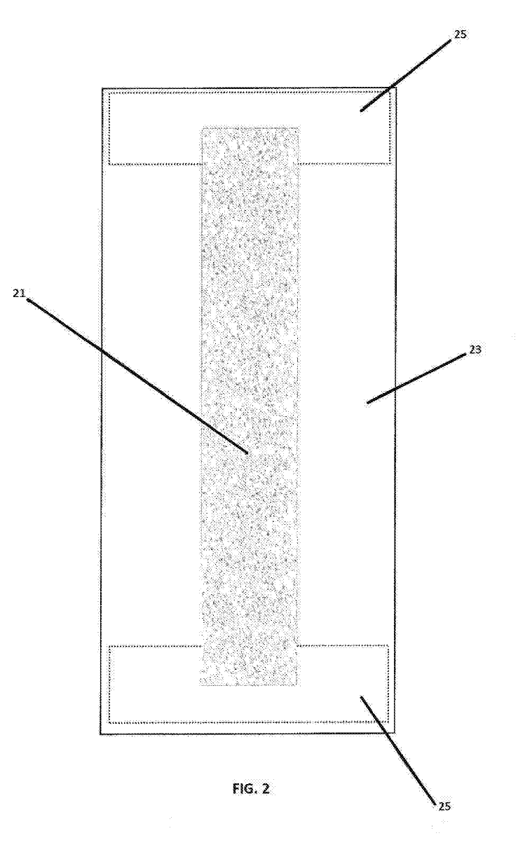

In a preferred embodiment, the thermoplastic composition contacts a component of the absorbent core directly. The thermoplastic composition can be applied to the core facing side of the top sheet and then brought together with the back sheet to directly overlap the absorbent core (either the core or the core wrap) in the final article (FIG. 1). Alternatively, the thermoplastic composition (FIG. 2, 25) can be applied to the core wrap (FIG. 2, 23) and then wrapped around the core (FIG. 2, 21). The thermoplastic composition can also be applied directly to the finished absorbent core (i.e. with or without core wrap), e.g. across the short ends of the core.

In another embodiment, the thermoplastic composition overlaps the absorbent core indirectly i.e. another material is between the absorbent core and the thermoplastic composition e.g. the thermoplastic composition is applied over the body facing side of the top sheet—overlapping the absorbent core situated beneath the top sheet.

The thermoplastic composition is applied in a full coat layer (i.e. a layer with 100% coverage) as a discrete shape, overlapping a portion of the core. The discrete shape is strategically located to decrease the likelihood of leaks in the desired location. The size, thickness, and location of the shape can be varied depending on the type of leak prevention desired. As an example, in some cases using a lower coat weight and decreasing some leaks may be desired, in other cases using a higher coat weight and decreasing all leaks may be desired.

The thermoplastic composition can overlap a portion, or the entire length of one side of the core (e.g. the back waist region), a portion, or the entire length of two sides of the core, or even a portion, or the entire length of all sides of the core. In one embodiment the thermoplastic composition comprises a rectangular shape overlapping a portion of one side of the core. In another embodiment, the thermoplastic composition forms a rectangular shape overlapping a portion of two sides of the core. In still another embodiment, the thermoplastic composition overlaps a portion of each side of the core (FIGS. 1, 3 and 5).

The thermoplastic composition is applied at a coat weight from about 1 gram per square meter (gsm) to about 50 gsm, from about 5 to about 40 gsm, from about 5 to about 20 gsm, or even from about 10 to about 20 gsm.

The application method to apply the thermoplastic composition is not particularly limited. Any application method can be used which results in a fill coat layer i.e. a layer with 100% thermoplastic composition coverage. The thermoplastic composition can be applied as a pre-coated film. The thermoplastic composition can be foamed. The thermoplastic composition can be applied in a molten state by non-contact or by contact application. The thermoplastic composition can be applied by use of a slot applicator or a high coverage spray system. One layer of thermoplastic composition can be used, alternatively two layers can be used one on top of the other. The applicator can be fed by either a gear pump, extruder or a constant pressure pump.

The leak barrier is useful in the production of a wide variety of disposable absorbent articles including, e.g., disposable diapers, adult incontinence products (e.g. briefs and pads), sanitary napkins, medical dressings (e.g., wound care products) bandages, surgical pads, pet training pads (e.g. puppy pads) and meat-packing products.

The invention will now be described by way of the following examples. All parts, ratios, percents and amounts stated in the Examples are by weight unless otherwise specified.

### EXAMPLES

### Test Methods

Test procedures used in the examples and throughout the specification, unless stated otherwise, include the following.

### Viscosity Test Method

Viscosity is determined in accordance with ASTM D-3236 entitled, “Standard Test Method for Apparent viscosity of Adhesives and Coating Materials,” (Oct. 31, 1988), using a Brookfield Thermoset viscometer Model RVDV 2 and a number 27 spindle. The results are reported in centipoise (cP).

### Adult Incontinence Brief Leak Test

Two finished adult incontinence briefs were acquired.

A leak barrier was added to one of them. The leak barrier was added to overlap the entire length of the back waist side of the absorbent core. The leak barrier consisted of a 15 gsm, 100 cm by 50 cm full coat of thermoplastic composition* on a spun bond non-woven. The leak barrier was pressed into place, on the outward facing side of the top sheet to overlap 4 centimeters of the core. Each adult incontinence brief was tested in the following way. *The thermoplastic composition used was a olefin based hygiene construction adhesive.

The adult incontinence brief was opened and laid flat on an angled board. The board was secured to give a 20 degree angle from the table top. The brief was secured to the board. The back waist portion of the brief was positioned on the non-elevated portion of the board. The board was wide enough and long enough to accommodate the adult incontinence brief (e.g. 80 cm by 65 cm).

A separatory funnel was positioned in a ring stand, at the elevated end of the brief, touching the top sheet of the absorbent article, approximately 2.54 cm behind and along the center line of the core structure, The funnel was filled with saline solution (0.9%). A catch pan was positioned under the diaper to catch all the fluid that leaked from the core at the non-elevated end. 250 milliliters (ml) of saline solution was discharged out of the separatory funnel at a rate of 7 ml/sec. This was repeated with an additional 250 ml. The intent was to simulate two typical adult voids that may occur overnight. For each article, the fluid that leaked from the non-elevated end was measured in ml.

TABLE ONE

| | Adult Incontinence | Adult Incontinence Product- |
|---|---|---|
| | Product-Control | Including leak barrier |
| Amount of fluid leaked | 50 | 0 |
| (milliliters) | | |

*The thermoplastic composition used was a olefin based hygiene construction adhesive.
Note:
The leak test could be adjusted to test diapers and light incontinence products by lowering the amount of fluid used in the test.

Note: The leak test could be adjusted to test diapers and light incontinence products by lowering the amount of fluid used in the test.

Other embodiments are within the claims.

What is claimed is:

1. A disposable absorbent article, comprising:
a. a top sheet,
b. a back sheet, and
c. an absorbent core disposed between the top sheet and the back sheet,
the top sheet, the back sheet, and the absorbent core forming a layered assembly held together with an attachment means, the disposable absorbent article further comprising a thermoplastic composition applied in a full coat layer as a discrete shape to the top sheet or to a component of the absorbent core and overlapping a portion of the absorbent core.

2. The disposable absorbent article of claim 1 wherein the thermoplastic composition overlaps a component of the absorbent core with direct contact.

3. The disposable absorbent article of claim 1 wherein the thermoplastic composition is applied to the core facing side of the top sheet.

4. The disposable absorbent article of claim 1 wherein the absorbent core comprises:
a. a core, and
b. a core wrap,
and the thermoplastic composition is applied to the core wrap prior to wrapping the core.

5. The disposable article of claim 1 wherein the attachment means is a hot melt adhesive composition.

6. The disposable absorbent article of claim 1 selected from the group consisting of an adult incontinence product, disposable diaper, sanitary napkin, bed pad, puppy pad and a medical dressing.

7. The disposable absorbent article of claim 1 further including a front waist region, a back waist region, and a crotch region disposed between the front waist region and the back waist region.

8. The disposable absorbent article of claim 7 selected from the group consisting of an adult incontinence brief and a disposable diaper.

9. The disposable absorbent article of claim 1 wherein the thermoplastic composition is applied to the absorbent core facing side of the top sheet.

10. The disposable absorbent article of claim 1 wherein the thermoplastic composition overlaps a portion of one side of the absorbent core.

11. The disposable absorbent article of claim 1 wherein the thermoplastic composition is applied at a coat weight of from about 5 to about 40 gsm.

12. The disposable absorbent article of claim 5 wherein the hot melt adhesive composition and the thermoplastic composition are the same.

13. The disposable absorbent article of claim 1 wherein the thermoplastic composition comprises a polymer selected from the group consisting of a styrene block copolymer and an olefin polymer.

14. The disposable absorbent article of claim 1 wherein the thermoplastic composition comprises at least 90% by weight of an olefin polymer.

15. The disposable absorbent article of claim 1 wherein the thermoplastic composition has a viscosity of from about from about 500 to about 25,000 cP at 177° C.

16. The disposable absorbent article of claim 7 wherein the thermoplastic composition comprises a rectangular shape overlapping a portion of the back waist region of the core.

17. A method of forming a leak barrier on a disposable absorbent article comprising the steps of:
a. applying a thermoplastic composition in a full coat layer of a discreet shape to the top sheet or to a component of the absorbent core, and
b. joining the top sheet to the back sheet while inserting the absorbent core,
such that the thermoplastic composition overlaps a portion of the absorbent core.

18. The method of claim 17 wherein the disposable absorbent article is selected from the group consisting of an adult incontinence brief and a disposable diaper.

19. The method of claim 17 wherein the discreet shape of a thermoplastic composition is applied in molten form with a slot coater.

70. An absorbent core comprising:
a. a core,
b. a core wrap, and
c. a thermoplastic composition,
wherein the thermoplastic composition is applied in a full coat layer of a discreet shape to the core wrap, so as to directly overlap a portion of the core when wrapped around the core.
